(19) United States
(12) Patent Application Publication
(10) Pub. No.: US 2009/0215166 A1
(43) Pub. Date: Aug. 27, 2009

(54) Cationic Polymer for Transporting Nucleic Acids in Cells

(75) Inventors: Achim Göpferich, Sinzing (DE); Miriam Breunig, Regensburg (DE); Uta Lungwitz, Regensburg (DE); Torsten Blunk, Pentling (DE)

Correspondence Address: BOWDITCH & DEWEY, LLP, 311 MAIN STREET, P.O. BOX 15156, WORCESTER, MA, 01615-0156, US

(73) Assignee: UNIVERSITAT REGENSBURG, REGENSBURG (DE)

(21) Appl. No.: 12/063,935

(22) PCT Filed: Aug. 16, 2006
(86) PCT No.: PCT/EP2006/008057

§ 371 Date: Nov. 7, 2008

(30) Foreign Application Priority Data
Aug. 17, 2005 (DE) 102005039154.0

(51) Int. Cl. C12N 15/85; C08F 26/02

(52) U.S. Cl. 435/320.1; 526/310

(57) ABSTRACT

The invention relates to a cationic polymer containing cationic oligomers cross-linkable by fissile linker sequences, wherein said cationic polymers form, together with nucleic acids, polyplexes and can be used for cell transfection and the invention is characterised in that the cationic polymer contains intracellular reductive or enzymatic fissile linker sequences.

The present invention relates to biologically degradable cationic polymers for transporting nucleic acids into cells. The invention relates in particular to cationic polymers of the type which are intracellularly degradable.

Transfecting or transferring genetic information by means of nucleic acids into human cells as well as animal cells (transfection) is a method often used today in biotechnology. At present, the development of effective and predominantly cell-compatible transfection systems is being vigorously pursued so that they may also be used in the treatment of diseases such as cystic fibrosis or cancer.

The transfer of nucleic acids into cells is therefore an important procedure for numerous medical and also scientific inquiries. In the field of gene therapy, attempts are being made, for example, to replace or exchange genes by the transfer of DNA into cells. In the field of research, a similar result is desired from the transfer of nucleic acids into cells and the intention is to influence the function or behaviour of cells either temporarily (transient) or permanently. Included among nucleic acids are, for example desoxyribonucleic acid (DNA), ribonucleic acid (RNA), siRNA, cyclic DNA (plasmids), antisense oligonucleotides and derivatives of all these substances which are known per se from the literature to a person skilled in the art.

A problem of this procedure is that most of the aforementioned substances or the derivatives thereof have a negative charge due to their chemical structure. This presents a drawback in negotiating the cell membrane which is usually composed of lipids. The rate of passage from the outside of the cell to the inside of the cell is either too slow or completely impossible in many cases for such large, charged molecules. It is for this reason that efforts are dependent on “associating” nucleic acids with other substances.

Viral vectors are particularly efficient for this purpose, as for example in cell cultures they are able to transfect virtually all cells of a population. However, they have serious disadvantages, of which a person skilled in the art is aware. In vivo, they have to some extent a huge immunising potential which has already resulted in fatalities in clinical studies (Marshall E. What to do when clear success comes with an unclear risk? Science 2002; 298: 510-511). Moreover, they harbour a not insignificant oncogenic risk (Sadelain M. Insertional oncogenesis in gene therapy: how much of a risk? Gene Ther 2004; 11: 569-573). In vitro, the problem frequently arises that viral vectors are awkward to handle, since a number of processing steps are necessary to obtain an efficient reagent. Although viral gene transfer is thus established today as an in vitro and in vivo transfection process, its applicability is restricted by high costs and high risks.

As an alternative to using viruses as carriers for nucleic acids, systems have become established which consist of nonviral components. In the past years, particular attempts have been made to compensate for the charge of nucleic acids. This is achieved by complexing nucleic acids with positively charged molecules and obtaining thereby neutral to positively charged aggregates. In an ideal case, these aggregates can become attached to the overall negatively charged cell membrane and ensure absorption into the cell.

Very many of these cationic compounds are known to a person skilled in the art (Han So, Mahato R I, Sung Y K, Kim S W. Development of biomaterials for gene therapy. Molecular Therapy 2000; 2: 302-317 and Nishikawa M, Huang L. Nonviral vectors in the new millennium: delivery barriers in gene transfer. Hum Gene Ther 2001; 12: 861-870). They include, inter alia, inorganic compounds, for example calcium salts, and equally organic compounds from the range of lipids or also polymers having molecular weights of more than 500 Da.

In particular in the field of polymers, relevant compounds are known which are able to transfect field acids into cells. These usually consist of monomers having functional groups which carry positive charges which compensate the negative charges of the nucleic acids. This produces so-called polyplexes which generally consist of particles having a size of a few to several thousand nanometres.

Polymers suitable for this type of complexing are, for example polyethylenimines poly(L-lysine), chitosan, polyvinylpyrrolidone (PVP) and polydimethylaminomethacrylate. The functional groups which charge these materials under the aforementioned conditions are preferably primary and secondary amino groups which are positively charged. The genetic information passes, for example by way of adsorptive endocytosis inside the cell and is there converted into the desired protein.

A disadvantage of these polycationic compounds is that they have a lower transfection efficiency than viral systems for transferring nucleic acids. Moreover, due to their charge, not only do they complex the compounds which are desired to be transfected into the cell, but also the nucleic acids which are naturally present in the cell. Consequently, it is observed that during the transfer of nucleic acids into cells, a considerable proportion of the cells often dies. In the worst case scenario, this toxicity can mean that no cells survive the transfer of the nucleic acids. In addition to the relatively poor transfection yield, this is a considerable disadvantage of polycationic compounds in respect of the transfer of nucleic acids. Moreover, based on such in vitro results, there is presently a great reticence in using nonviral transfection systems in vivo.

A principle which could reduce the cytotoxicity of such systems is the use of intracellularly degradable polymers. Attempts are presently being made to implement this strategy by cross-linking branched polyethylenimines with acid-labile linkers. A few systems of this type have already been patented (U.S. Pat. No. 6,312,727, U.S. Pat. No. 6,652,886 and U.S. Pat. No. 6,794,189). The approaches described therein, however, involve a few problems:
1. Many of these polymers already decompose autocatalytically in a neutral environment and thus prevent a targeted release.
2. If relatively stable derivatives are used, after the polyplex has been absorbed by endocytosis, efforts are dependent on a sufficiently acidic pH in the endolysosome to achieve degradation of the polymer, the high buffer capacity of the polymer making this difficult.
3. As soon as the degradation is complete, the nucleic acid is at least partly released from the polyplex and is thus vulnerable to DNA-cleaving enzymes of the endosomolytic vesicles.

To allow a programmable dissociation which is independent of a change in pH, it is basically possible to cross-link polycationic compounds with intracellularly reducible linkers. In so doing, the polymer chain is cleaved independently of the pH, and it releases the nucleic acid. An approach of this type was followed using longer-chain polylysine derivatives for reversible polyplex stabilisation (R. C. Carlisle, T. Etrych, S. S. Briggs, J. A. Preece, K. Ulbrich, L. W. Seymour; Polymer-coated polyethylenimine/DNA complexes designed for triggered activation by intracellular reduction. J. Gene Med. (2004); 6; 337-344; M. L. Read, K. H. Bremner, D. Oupicky, N. K. Green, P. F. Searle, L. W. Seymour; Vectors based on reducible polycations facilitate intracellular release of nucleic acids. J. Gene Med. (2003); 5; 232-245; D. Oupicky, R. C. Carlisle, L. W. Seymour; Triggered intracellular activation of disulfide crosslinked polyelectrolyte gene delivery complexes with extended systemic circulation in vivo. Gene Ther. (2001); 8; 713-724).

However, polylysines are cationic polymers which, as shown many times, have a low endosomolytic activity and which clearly differ from materials with a high endosomolytic activity, for example polyethylenimines (Sonawane N D, Szoka F C, Verkman A S. Chloride accumulation and swelling in endosomes enhances DNA transfer by polyamine-DNA polyplexes. J Biol Chem 2003; 278: 44826-44831).

It is possible to make a distinction experimentally between substances which have a low endosomolytic activity and those which have a high endosomolytic activity. The potential to transfect nucleic acids into cells can only be increased in the case of substances which have a low endosomolytic activity by adding substances such as saccharose, quinine, viral proteins and other substances familiar to a person skilled in the art to destabilise membranes (in particular of endosomes and lysosomes). This use of membranolytic agents is disadvantageous, as they are not readily compatible with the cells.

Oligomers of polyethylenimine, for example, have a particularly high endosomolytic activity which could not be increased in a transfection experiment by adding lysomotropic substances (Ciftci K, Levy R J. Enhanced plasmid DNA transfection with lysosomotropic agents in cultured fibroblasts. Int J Pharm 2001; 218: 81-92); (Breunig M, Lungwitz U, Liebl R, et al. Gene delivery with low molecular weight linear polyethylenimines. J Gene Medecine. In press.). A characteristic of substances having a high endosomolytic activity is therefore that the membranolytic potential cannot be significantly increased by adding additional membranolytic agents, for example lysomotropic substances.

The invention relates to cationic polymers, also called polycations, which are able to complex nucleic acids, have a high endosomolytic activity and are degradable in particular in an organism, preferably in cells and most preferably in cytoplasm, the degradation products having substantially no cytotoxicity.

This object is achieved according to the invention by a cationic polymer which is composed at least of cationic oligomers, the cationic oligomers being linked by linkers which are cleaved under physiological conditions.

The present invention relates in particular to such cationic polymers which are able to transfect cells with nucleic acid, the cationic polymer having a higher transfection efficiency compared to the oligomer.

According to a further aspect of the present invention, linkers in particular are used which can be cleaved intracellularly, the cleavage preferably taking place enzymatically or reductively and the cleavage is preferably independent of a change in the pH.

The intracellular cleavage can take place, for example in the endosome or in the cytosol, cleavage in the cytosol being preferred.

The present invention is described in the following with reference to the accompanying drawings.

The embodiments shown in the drawings are particularly preferred embodiments of the invention which are particularly suitable for explaining the present invention, without thereby restricting the present invention. In the drawings:

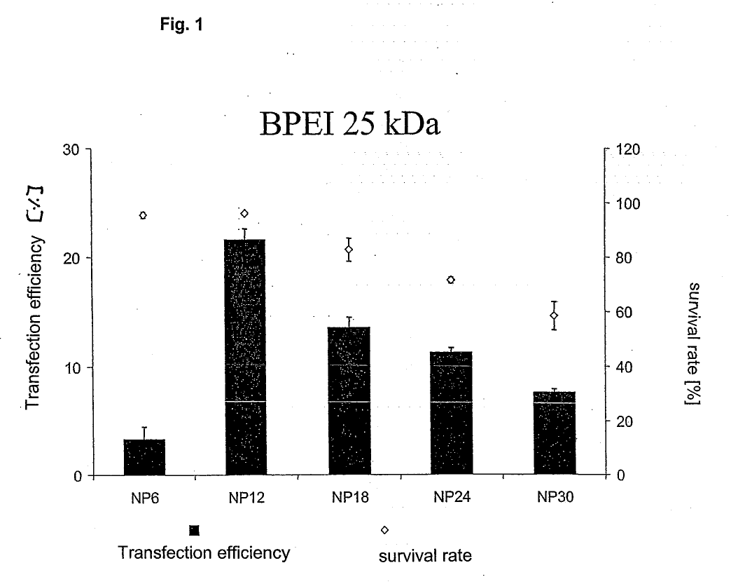

FIG. 1 shows a diagram with the transfection efficiency and cell survival rate as a function of the molecular weight on the example of branched polyethylenimine (BPEI);

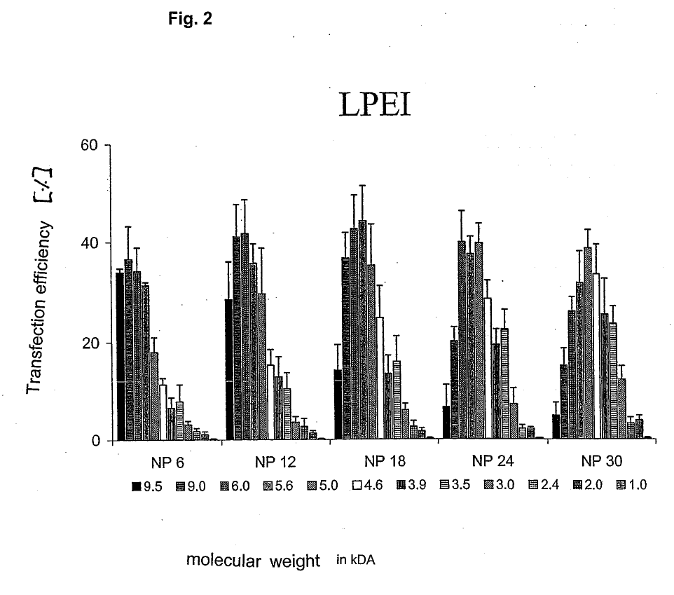

FIG. 2 shows a diagram with the transfection efficiency of linear polyethylenimine (LPEI) as a function of molecular weight;

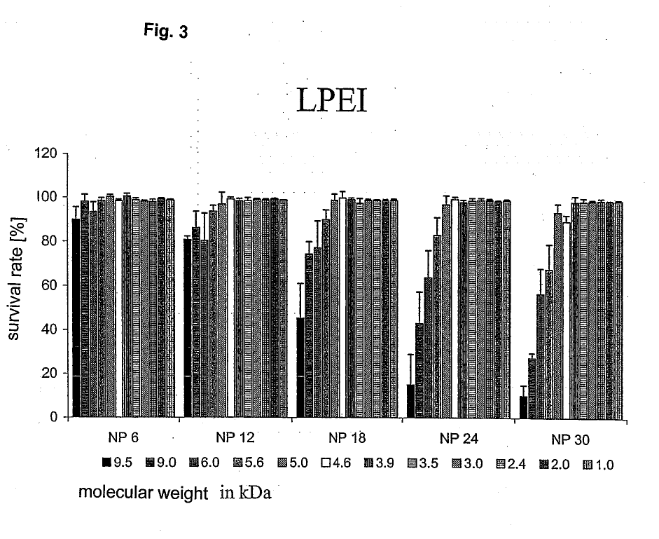

FIG. 3 shows a diagram of the cell survival rate during transfection with LPEI according to FIG. 2;

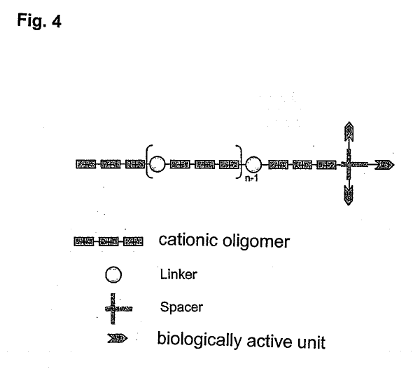

FIG. 4 schematically shows the construction of an embodiment of the cationic polymer according to the invention;

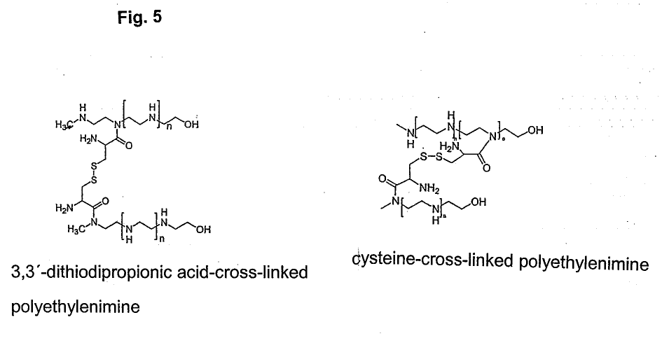

FIG. 5 shows the structural formula of two embodiments preferred according to the invention of cross-linked cationic polymers having different linkers;

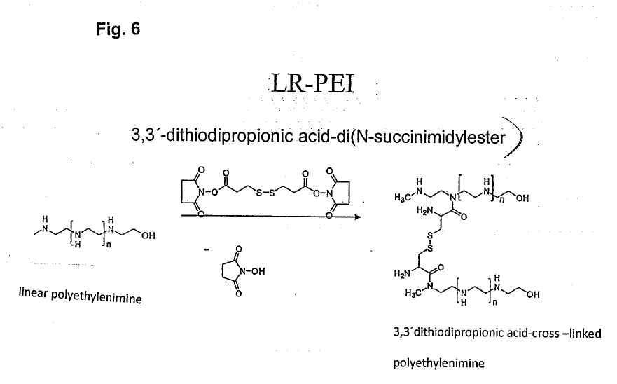

FIG. 6 shows the reaction scheme of the preparation of the cationic polymer preferred according to the invention, according to FIG. 5 with Lomant's reagent (LR);

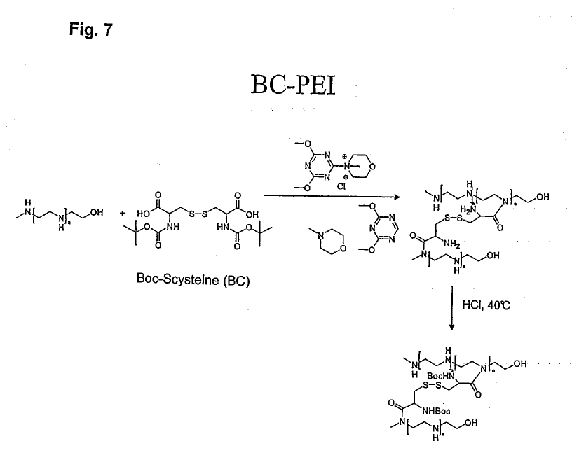

FIG. 7 shows the reaction scheme of the preparation of the further cationic polymer preferred according to the invention, according to FIG. 5 with Boc-cysteine (BC);

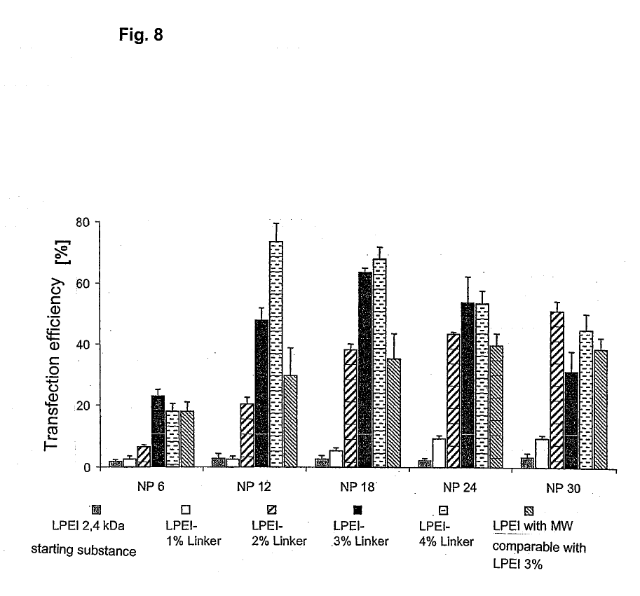

FIG. 8 shows a diagram of the transfection efficiency of cationic polymers according to the invention as a function of the cross-linking rate; and

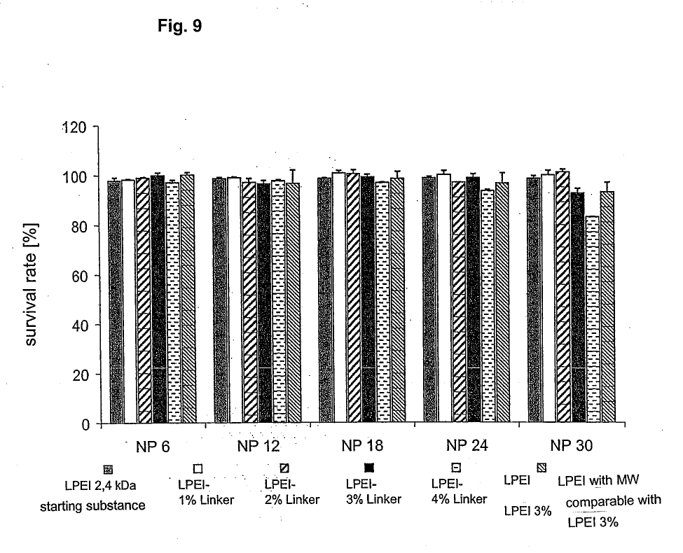

FIG. 9 shows a diagram of the cell survival rate of the cationic polymers according to FIG. 8.

Polycations generally exhibit the behaviour that as the molecular weight increases, so does the ability to transport nucleic acids into cells or to transfect them. During experiments on cytotoxicity and transfection efficiency, the present inventors have found from the example of branched polyethylenimine that the polymers become increasingly toxic as the ratio of polymer (expressed as nitrogen content in mol N) to nucleic acid (expressed as phosphorus content in mol P) increases, i.e. with an increasing N/P ratio. In other words, polyplexes which, as shown in FIG. 1, contain increasing quantities of polyethylenimine, become increasingly toxic (see also Godbey W T, Wu K K, Mikos A g. Size matters: molecular weight affects the efficiency of poly(ethylenimine) as a gene delivery vehicle. J Biomed Mater Res 1999; 45: 268-275 and Godbey W T, Wu K K, Mikos A G. Poly(ethylenimine)-mediated gene delivery affects endothelial cell function and viability. Biomaterials 2000; 22: 471-480).

A branched polyethylenimine having a molecular weight of 25 kDa was used as the starting polymer in the experiment shown in FIG. 1.

As a comparison, the present inventors thereupon investigated the linear compounds and surprisingly found that linear PEI (LPEI) has a similarly high transfection efficiency (FIG. 2) as branched PEI, but exhibits a substantially lower toxicity as a function of molecular weight (FIG. 3). The molecular weights of the polymers on which the individual experiments are based are given in FIGS. 2 and 3.

Molecules having a molecular weight of in particular less than 600 Da proved to be nontoxic (Godbey W T, Wu K K, Mikos A G. Poly(ethylenimine)-mediated gene delivery affects endothelial cell function and viability. Biomaterials 2000; 22: 471-480).

Polyplexes of LPEI having a molecular weight of less than 3900 Da proved to be nontoxic in every N/P ratio, but were still able to transfect cells.

It could thus be shown for the first time that it is possible to synthesise polymers which are able to transfect nucleic acids into cells, independently of the N/P ratio, without the cells dying off. It was then surprisingly found that by suitably linking these oligomers to produce polymers, the transfection efficiency increases, without giving rise to concurrent toxic reactions.

On the basis of the above finding, a polymer platform can be provided according to the invention which allows the transfection efficiency and toxicity to be uncoupled from the molecular weight of the polymers, in that nontoxic oligomers are linked to form polymers. For this purpose, a polymer system was developed which contains several components and which meets the requirements imposed on an agent of this type for use in vivo and also in vitro.

Necessary constituents of the cationic polymers according to the invention are cationic oligomers which, taken on their own, do not exhibit a satisfactory transfection as they do not have an adequate molecular weight, but in their favour are nontoxic.

The cationic oligomers are linked by means of linkers to form the polymer according to the invention. The linkers used according to the invention are capable, after being absorbed into the cell by means of endosomes, of escaping therefrom due to their endosomolytic characteristics. The linkers also increase the molecular weight. On the one hand, this ensures an improved complexing of nucleic acids. On the other hand, the cationic polymer is degraded inside the cell, to again produce the nontoxic cationic oligomers, due to the chemical environment prevailing in the cell combined with the chemical characteristics of the linkers.

The cationic polymer according to the invention can optionally be linked with a biologically active unit. This unit serves to improve the efficiency of the polymer, in particular with respect to the resulting transfection efficiency, by means of cellular structures or mechanisms. The biologically active unit can be coupled with the cationic polymer either directly or by means of a spacer.

The materials which can be derived from this structural plan and are the object of the invention described here, consist at least of the cationic polymer with oligomers cross-linked by means of linkers. The biologically active unit is optional and can be bound to the cationic polymer selectively with or without spacers. The structure of the cationic polymer according to the invention together with the biologically active unit, which in this case is bound by means of a spacer, is shown schematically in FIG. 4.

The individual components of the polymer platform are described in detail in the following.

a) Cationic Oligomers

Suitable as cationic oligomers for the cationic polymer of the present invention are basically all chemical compounds which, under physiological conditions, i.e. pH 4 to 8 and in particular at a pH of approximately 7.4, carry at least one positive charge and can be linked to form the cationic polymer by means of a linker. In particular for the present invention, monomer units can be used which are able to form cationic oligomers, the oligomers substantially being nontoxic to cells and forming, by linking with the linkers used according to the invention, cationic polymers which have an improved transfection efficiency compared to the oligomer.

The cationic oligomers can be homo-oligomers or co-oligomers of two or more different monomer units.

Examples of suitable oligomers according to the invention can contain up to 700 monomer units. More preferably they contain up to 200 monomer units, in particular up to 120 monomer units, and further preferred up to 60 monomer units.

It is understood that the number of monomer units in the oligomer and thus the molecular weight of the oligomer can vary as a function of the monomer unit which is specifically used in each case and should be selected such that the resultant oligomer is substantially nontoxic in the cell.

More preferably, the oligomer used according to the invention contains more than 10 and in particular more than 12 monomer units.

Examples of suitable monomer units include ethylene amine, imdazole, lysine, arginine and histidine. Further examples of suitable oligomers include oligomers based on chitosan, vinyl alcohol or oligomers of 2-dimethylaminomethacrylate.

An oligomer preferred according to the invention is linear polyethylenimine (LPEI) having a molecular weight of up to 30000 Da. Oligomers of LPEI having a molecular weight of up to 8000 and up to 5000 are more preferred and oligomers of LPEI having a molecular weight of up to 2500 Da are most preferred. It was also possible to obtain good results with an LPEI oligomer having a molecular weight of up to 1000 Da.

Oligomers of different compositions can be used for the cationic polymer. Suitable examples here include cationic polymers with oligomers based on branched and linear PEI. Further examples include combinations of oligolysine, oligoarginine, oligohistidine, oligoimidazole or oligomers with these molecules or monomers in the side chains.

Further suitable combinations are cationic polymers with oligomers of LPEI optionally combined with branched PEI as well as combinations thereof with the aforementioned oligomer.

It could be shown in cell culture models that polyplexes of the cationic polymer according to the invention and nucleic acid are even nontoxic in the case of an N/P ratio of 60 and still transfect the cells efficiently.

Thus, for the purpose of the present invention, an N/P ratio of not more than 60 and in particular not more than 30 is particularly suitable.

b) Linkers

The linkers operate as predetermined breaking points between the oligomers in the cationic polymer. Characteristics of the linkers are functional groups which are cleaved under physiological conditions and thus disintegrate into two or more molecular parts. This leads to the cationic polymer, produced by the linker, breaking between the oligomers.

The breaking is not only restricted to covalent chemical bonds, but also includes, for example complexes which disintegrate in the cell, or other chemical bonding mechanisms. An example of this is that linkers which consist of complex ligands and a central molecule, for example a cation, disintegrate by exchanging the central molecule.

The linkers are able to increase the molecular weight of the oligomer to the cationic polymer by factors of up to 1,000,000. An increase of the molecular weight by factors of up to 100 is preferred, and in particular by factors of up to 10.

The linker is preferably only cleaved inside the cell and in so doing, reverts in particular to established reactions or characteristics which have been described for the plasma. These can be enzymatic cleavages, for example those produced by peptidases or esterases, or redox reactions, for example the reduction of disulphides, or disulphide exchange and others known to a person skilled in the art from the literature.

The preferred half-life of these reactions is up to 24 hours. More preferred are half-life periods of up to 2 hours and most preferred are reactions with a half-life of up to 30 minutes.

Examples of linkers are those which contain disulphides. The reduction of the disulphide predetermined breaking points by cell-endogenous glutathione leads to the decomposition of the polymer into shorter, nontoxic constituents.

Specific examples of suitable linkers are glutathione, dimers of cysteine, Boc-cysteine or 3,3′-dithiodipropionic acid oximide.

According to the present invention, the proportion of linker in the cationic polymer is preferably 10% (m/m) or less and particularly preferably at least 2% (m/m).

A more preferred quantity range is from at least 2% (m/m) up to 6% (m/m), in particular from at least 2% (m/m) to 4% (m/m).

According to the invention, the oligomers, of which the cationic polymer is composed, are linked with one another by the linkers. This means that the linkers are present distributed within the entire polymer structure. During the formation of the polyplexes, the linkers are thus likewise present over the entire formed complex and thus also inside the complex.

c) Biologically Active Unit

Ligands for receptors on the surfaces or inside specific cells can be the biologically active unit. Examples here include nuclear localisation sequences (Dean D A, Strong D D, Zimmer W E. Nuclear entry of nonviral vectors. Gene Ther 2005; 12: 881-890), the TAT peptide (Gupta B, Levchenko T S, Torchilin V P. Intracellular delivery of large molecules and small particles by cell-penetrating proteins and peptides. Adv Drug Deliv Rev 2005; 57: 637-651), Transferrin (Kursa M, Walker G F, Roessler V, Ogris M, Roedl W, Kircheis R, Wagner E. Novel Shielded Transferrin-Polyethylene Glycol-Polyethylenimine/DNA Complexes for Systemic Tumor-Targeted Gene Transfer. Bioconjug Chem 2003; 14: 222-231), Antibodies or their fragments (Kircheis R, Kichler A, Wallner G, Kursa M, Felzmann T, Buchberger M, Wagner E. Coupling of cell-binding ligands to polyethylenimine for targeted gene delivery. Gene Ther 1997; 4: 409-418.

Further specific suitable examples of biologically active units are cytokines, growth factors, for example EGF, oligo- and polysaccharides, mono- and disaccharides such as lactose, galactose, mannose and glucose, as well as folates.

Moreover, all substances are suitable which, as ligands, bind to receptors of cells. Substances of this type are known to a person skilled in the art from the field of medical chemistry.

Further examples include peptides and proteins which are capable of interacting with proteins of the cell, and substances, for example from the group of lipids or charged compounds which are able to interact with the cell surface due to their physical and chemical characteristics.

d) Spacers

The spacer is expendable per se, but can be advantageous in many respects. For example, it can be used to produce a space between the cationic polymer and the biologically active unit. This can be advantageous, for example in preventing undesirable interactions, for example due to electrostatic interactions. Furthermore, the spacer can be used to give the entire molecule an orientation so that, for example, a plurality of these molecules can be stored together with nucleic acids to produce colloids with a defined structure. Consequently, this can mean, for example that the biologically active unit does not remain hidden inside the colloids where it cannot interact with the biological system.

Depending on requirements, the spacer can have different chemical structures and in principle can also be low molecular with a molecular weight of up to 400 Da. However, polymers having molecular weights of up to 600000 Da are preferred. More preferred are polymers with a molecular weight of up to 20000 Da. Polymers with a molecular weight of between 500 and 5000 Da are most preferred.

The charge of the spacer does not necessarily have to be neutral under physiological conditions. However, spacers which do not carry a net charge are preferred. The spacer can also be multifunctional. Thus, it can contain branches, as shown in FIG. 4. This makes it possible, for example to increase the density of the biologically active units, or to bind various units of this type having different tasks.

Specific examples of spacers include polyethylene glycols, pluronics, polysialic acid, hyaluronic acids, polyacrylic acids, dextranes, transferrin, poly(N-(2-hydroxpropyl)methacrylamides and derivatives of these substances.

The cationic polymer according to the invention and the oligomers can be prepared according to polymerisation reactions known per se. The linker can be added as an independent molecule. The linker can also be produced by reacting corresponding functional groups on the oligomer during the linking to produce the cationic polymer. Processes of this type are generally known per se to a person skilled in the art.

The present invention relates to a new cationic polymer at least consisting of cationic oligomers which are cross-linked by means of preferably intracellularly cleavable linkers. By complexing with nucleic acids, polyplexes are produced which are absorbed by endocytosis into a large number of cells and thus they transport the genetic information inside the cell. Characteristic of the cationic polymer according to the invention is that biologically relevant quantities of nucleic acids, i.e. sufficient quantities to produce a biological effect, can be complexed and transported. Compared to other known transfection reagents, the preferably intracellularly cleavable linkers used according to the invention can achieve a higher efficiency in the same model, without thereby having to accept toxic effects. The present invention thus relates to biodegradable cationic polymers which allow a use in vitro and in vivo on account of their low toxicity.

The application possibilities of the cationic polymers according to the invention are widespread. Apart from the transfer of nucleic acids into cells, for example for transfection purposes, the cationic polymers can be used for embedding nucleic acids into other materials; thus, for example into porous polymer matrices consisting of polymers or lipids which are used as cell carriers in tissue engineering. A further application possibility is the use of the cationic polymers for the preparation of biodegradable polyelectrolyte multilayers, as obtained, for example for the production of films, microparticles or nanoparticles by the layer-by-layer (LBL) method. LBL films of this type show great promise for the production of DNA or RNA microarrays. Microparticles and nanoparticles which contain nucleic acid and are produced from these materials are suitable as DNA and RNA inoculants. In particular for immunisiation against AIDS, excellent therapeutic possibilities are emerging from the transfection of nucleic acids into the cells of the immune system.

As a result of increasing the degree of cross-linking between the oligomers used according to the invention, it is possible to obtain hydrogels which are suitable, when applied locally, for the controlled release of nucleic acids into tissues over relatively long periods of time.

The cationic polymers according to the invention can also be used to coat other materials to thus anchor nucleic acids or other polyanions to the surface. This is of particular interest in the case of nanoparticles for the target-oriented administration of medicaments (drug targeting). Particles of this type can also be, for example semiconductor crystals or magnetic materials which may also be easily detected on site.

### EXAMPLES

### Determination of Molecular Weight:

The weight was determined by size exclusion chromatography (SEC).

The following procedure was carried out to determine the molecular weight of LPEI:

20 mg of LPEI*HCl were dissolved in 1.0 ml of twice distilled water (ddH₂O), then filtered using a 0.2 μm polyethane sulphonic acid membrane filter. For chromatography, the temperature of a Noverna 300 C SEC column (10 μm, 8×300 mm, polymer standard service, Mainz) was controlled at 40° C., at a flow rate of 1 ml/min and 0.15 M NaCl were used as eluent.

The relative Mn, Mw and Mw/Mn of LPEI was calculated between 1.05 kDa and 340.5 kDa (polymer standard service, Mainz) using the elution volume of dextrane standard.

The molecular weights of the further compounds were determined analogously.

### 1. Synthesis of Cationic Oligomers Using the Example of Linear Polyethylenimine (LPEI)

1) Synthesis of Poly(2-ethyl-2-oxazoline):

2-ethyl-2-oxazoline and acetonitrile were dried under vacuum by distillation over calcium hydride (0.5 g/l), p-toluene sulphonic acid methylester as initiator. For the synthesis of poly(2-ethyl-2-oxazoline) having a molecular weight of 5700, 2-ethyl-2-oxazoline (monomer) was reacted with p-toluene suplhonic acid methylester (initiator) in a ratio of 58:1. 7 ml of acetonitrile per 1 ml of oxazoline were introduced as solvent and the initiator was dissolved therein. The required quantity of 2-ethyl-2-oxazoline was added using a syringe under an inert gas atmosphere and the reaction mixture was stirred at 90° C. under reflux. The reaction was stopped after 6 days and the polymer was precipitated in ice-cooled diethylether. The success of the reaction was verified by an ¹H-NMR spectrum (600 MHz) and the molecular weight and the molecular weight distribution were verified by SEC.

2) Synthesis of Linear Polyethylenimine:

Poly (2-ethyl-2-oxazoline) was mixed with an excess of 6N hydrochloric acid and acid hydrolysed under reflux at 100° C. for 48 hours. After removing the excess of volatile hydrochloric acid, the residue was absorbed in water and LPEI was precipitated with concentrated sodium hydroxide solution. The precipitate was washed by centrifugation with water up to a neutral reaction to remove excess sodium hydroxide solution and propionate formed during the reaction.

The conversion into LPEI was verified by ¹H-NMR (600 MHz) and the molecular weight and the molecular weight distribution were determined by SEC.

### EXAMPLE 2

Cationic Polymer of 3,3′-dithiodipropionic Acid—Cross-Linked Linear Polyethylenimine: (FIGS. 5 and 6)

Linear polyethylenimine was dried under vacuum at 60° C. and dissolved in 20 ml of dichloromethane. 3,3′-dithiodipropionic acid-di(N-succinimidylester) was dissolved in dichloromethane and added dropwise over a period of 30 minutes into the LPEI solution heated to 45° C. Different degrees of cross-linking are produced by the addition of 1-4% dithiodipropionic acid-di(N-succinimidylester) and diisopropylamine per ethylene amine unit. The reaction mixture was stirred under reflux overnight at 45° C. After termination of the reaction, dichloromethane was removed under evaporation. The residue was dissolved in 2N hydrochloric acid and the volatile constituents were removed by evaporation. The residue was dissolved in water and the polyamine was precipitated with concentrated sodium hydroxide solution. The precipitate was washed by centrifugation to a neutral reaction to remove excess sodium hydroxide solution and the resultant N-hydroxysuccinimide.

The cross-linked PEI was dried at 60° C. under vacuum and the conversion was verified by ¹H-NMR in CDCl₃ (600 MHz). The molecular weight and molecular weight distribution were determined by SEC.

### EXAMPLE 3

### Cationic Polymer of Cysteine and Linear Polyethylenimine: (FIGS. 5 and 7)

Linear polyethylenimine was dried under vacuum at 60° C. and dissolved in 10 ml of ethanol. 4-(4,6-dimethoxy[1.3.5] triazin-2-yl) 4-methylmorpholiniumchloride hydrate (DMT MM) was dissolved in 4 ml of ethanol and added to the solution of Boc-cysteine (BC) in 2 ml of ethanol. After 30 min, the LPEI solution was added and the reaction mixture was stirred overnight at room temperature. Different degrees of cross-linking are produced by the addition of 3-8% BC and DMT MM per ethylene amine unit. After termination of the reaction, the mixture was concentrated to dryness and the residue was dissolved in 2N hydrochloric acid and shaken for 1 hour at 40° C. After removing the excess hydrochloric acid, the residue was dissolved in water and the polyamine was precipitated with concentrated sodium hydroxide solution. The precipitate was washed by centrifugation to neutral reaction to remove excess sodium hydroxide solution and the resultant water-soluble by-products.

The cross-linked PEI was dried under vacuum at 60° C. and the conversion was verified by ¹H-NMR in CDCl₃ (600 MHz). The molecular weight and molecular weight distribution were determined by SEC.

### EXAMPLE 4

Transfection of CHO-K1 Cells with Plasmidic DNA:

The polyplexes for transfection were prepared from 2 μg plasmid DNA (p-EGFP-N1) code for enhanced green fluorescent protein (EGFP) and a corresponding amount of polymer to achieve an NP ratio of 6 to 30. For this, the polymer solution was directly pipetted to the DNA solution, then vortexed for 20 sec and incubated at room temperature for 20 minutes.

CHO-K1 cells were seeded into 24-well plates with a starting cell density of 38000 about 18 hours before transfection. Immediately before the polyplex addition, the cells were washed with PBS buffer and mixed with 900 μl fresh culture medium (serum-free). The polyplexes which were produced were pipetted directly into the culture medium. After 4 hours, polyplexes not absorbed by the cells and the culture medium were removed by suction and replaced by fresh culture medium. After a further 24 hours, the cells were trypsinised, washed with PBS buffer and the transfection efficiency and survival rate were determined by flow cytometry. The GFP positive cells were detected after being excited by laser light of 488 nm at 530 nm. Dead cells were stained with propidium iodide and detected in the same test setup at a wavelength of more than 670 nm. In total, 20000 events were counted. The GFP positive cells correspond to the transfection efficiency, the propidium iodide negative cells are expressed in the survival rate.

The results are shown in FIGS. 8 and 9.

1. A cationic polymer at least composed of cationic oligomers, wherein the cationic oligomers are linked via linkers, which are fissile at physiological conditions, wherein the cationic oligomers have at least one positive charge at physiological conditions at a pH-value in a range of from 4 to 8, and wherein the oligomer is composed of at least 23 monomer units.

2. The cationic polymer according to claim 1, wherein the fission takes place independently from a change of the pH-value.

3. The cationic polymer according to claim 1, wherein the fission takes place intracellular reductively or enzymatically.

4. The cationic polymer according to claim 1, wherein the oligomer is linear polyethylene imine.

5. The cationic polymer according to claim 4, wherein the oligomer of linear polyethylene imine is in combination of other oligomers.

6. The cationic polymer according to claim 1, wherein the amount of linkers does not exceed 10% (m/m).

7. The cationic polymer according to claim 1, wherein the cationic polymer is further linked to a biologically active unit for recognizing organelles or cells.
